(19) United States
(12) Patent Application Publication
(10) Pub. No.: US 2006/0241370 A1
(43) Pub. Date: Oct. 26, 2006

(54) Medical x-ray imaging workflow improvement

(76) Inventors: George Kramp, Elmhurst, IL (US); John Baumgart, Hoffman Estates, IL (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/237,556

(22) Filed: Sep. 28, 2005

Related U.S. Application Data
(63) Continuation-in-part of application No. PCT/US05/10864, filed on Mar. 30, 2005.

(51) Int. Cl. A61B 5/05

(52) U.S. Cl. 600/407

(57) ABSTRACT

A method and system reduce radiation exposure by panning and zooming a first image of a patient acquired by an x-ray imaging system rather than using continuous radiation fluoroscopy. A first image of a region of interest is initially acquired. Subsequently, the patient may be repositioned with respect to the imaging system or a component of the imaging system may be repositioned with respect to the patient. The first image may be automatically panned on a display in response to the repositioning. A collimator may be adjusted to select a coverage area of the collimator. The first image may be automatically resized on the display based upon the size of the selected coverage area. An optimal imaging device parameter also may be selected based upon the selected coverage area. A second image may be acquired using the imaging system and automatically resized to substantially fill the display.

### PRIORITY CLAIM TO RELATED APPLICATION

This is application is a continuation-in-part of and claims priority under 35 U.S.C. § 120 to PCT/US05/10864 filed on Mar. 30, 2005, entitled Exact Volume Imaging Involving Multiple Partial Scans, having attorney ref. no. 2004P05290US01, which is incorporated by reference in its entirety herein.

### FIELD OF THE INVENTION

The present invention relates generally to imaging systems. More specifically, the present invention relates to methods and systems for managing workflow to reduce the exposure of a patient to radiation during an x-ray imaging procedure.

### BACKGROUND

Digital x-ray imaging systems include C-arm volume imaging systems. The x-ray C-arm and patient may be repositioned with respect to each other during a medical procedure. Typically, the x-ray imaging systems operate in a fluoroscopic mode during the movement between positions in order to correctly reposition the C-arm.

Fluoroscopy is a technique that a radiologist or other technician uses during many diagnostic and therapeutic radiologic procedures to observe internal bodily images to assist with either the diagnosis or treatment of the patient. More specifically, a radiologist may obtain real time x-ray images of a patient using fluoroscopy. The real time x-ray images may be observed on a monitor for evaluation.

In a conventional x-ray imaging procedure, a radiologist will acquire a first image of the patient using an x-ray imaging system. Subsequently, the radiologist will reposition the patient to a second position determined by the fluoroscopy. A second image of the patient may then be acquired at the second position. However, operation of the x-ray imaging system in the fluoroscopic mode may expose the patient to continuous low level radiation during repositioning.

### BRIEF SUMMARY

A method and system may reduce the exposure of a patient to radiation during an x-ray imaging procedure. A first image may be acquired by an x-ray imaging system. Subsequently, the patient and/or a component of the imaging system may be repositioned with respect to the other. The first image may be automatically panned on a display based upon the repositioning. A coverage area of a collimator may be selected by adjusting the collimator. The first image may be automatically resized on the display based upon the selected coverage area of the collimator. An imaging device parameter also may be automatically selected based upon the selected coverage area. A second image may be acquired using the x-ray imaging system. The second image may be automatically resized to substantially fill the display. By tracking the relative position changes and adjusting the first image accordingly, fewer or no fluoroscopy images are needed before taking the second image.

In one embodiment, a method reduces the exposure of a patient to radiation during an x-ray imaging procedure. The method includes acquiring a first image of a region of the patient using an imaging system, repositioning the patient with respect to a component of the imaging system or the component of the imaging system with respect to the patient, and automatically panning the first image on a display based upon the repositioning. The method also may include acquiring a second image of the region using the imaging system.

In another embodiment, a method reduces the exposure of a patient to radiation during an x-ray imaging procedure. The method includes acquiring a first image of a region of the patient using an imaging system, repositioning the patient with respect to a component of the imaging system or the component of the imaging system with respect to the patient, selecting a selected coverage area of radiation of the imaging system, and automatically resizing the first image on a display based upon the selected coverage area. The method also may include acquiring a second image of the region using the imaging system.

In another embodiment, a data processing system reduces the exposure of a patient to radiation during an x-ray imaging procedure. The system includes a display operable to show a first image of a region of the patient obtained using an imaging system and a data processor connected with the display operable direct the panning or resizing of the first image on the display based upon the movement of the table or the C-arm.

In yet another embodiment, a computer-readable medium having instructions executable on a computer stored thereon is provided. The instructions include displaying a first image of a region of a patient obtained using an imaging system on a display and panning the first image on the display in response to movement of a table or a C-arm. The instructions also may include resizing the first image on the display in response to an adjustment of a coverage area of a collimator and displaying on the display a second image of the region obtained using the imaging system after the adjustment of the coverage area.

Advantages will become more apparent to those skilled in the art from the following description of the preferred embodiments which have been shown and described by way of illustration. As will be realized, the system and method are capable of other and different embodiments, and their details are capable of modification in various respects. Accordingly, the drawings and description are to be regarded as illustrative in nature and not as restrictive.

### BRIEF DESCRIPTION OF THE DRAWINGS

The present invention will become more fully understood from the detailed description given herein below and the accompanying drawings which are given by way of illustration only, and are not limitative of the present invention, and wherein:

FIG. 1 is an exemplary image of a region of a interest;

FIG. 2 is an exemplary method for reducing the exposure of a patient to radiation during an x-ray imaging procedure;

FIG. 3 is an exemplary view of automatically panning the first image acquired by the imaging system;

FIG. 4 is an exemplary view of automatically zooming an image acquired by the imaging system; and

FIG. 5 is an exemplary data processing system that reduces the exposure of a patient to radiation during an x-ray imaging procedure.

### DETAILED DESCRIPTION

A method and system may reduce the amount of radiation that a patient is exposed to during a radiographic procedure. The method and system reduce radiation exposure by automatically panning and zooming the first image acquired by an x-ray imaging system rather than employing continuous radiation fluoroscopy.

The method and system involve a first image being acquired by an x-ray imaging system. Subsequently, the patient and/or the imaging system, or a component thereof, may be repositioned with respect to each other. The first image may be automatically panned across a display in response to the repositioning.

After which, the coverage area of a collimator may be adjusted to a selected coverage area. The first image may be automatically resized or reshaped on the display based upon the selected coverage area. Additionally, an imaging device parameter may be automatically selected based upon the selected coverage area to enhance the visual quality of the first image on the display. A second image may be acquired using the x-ray imaging system. The second image may be automatically resized to substantially fill the display.

Conventionally, internal images of patient may be obtained by an imaging system, such as a digital x-ray imaging system that includes a C-arm volume imaging system. A C-arm imaging system has a source and detector that are 180 degrees opposite of each other at the ends of a C-arm. The C-arm is capable of being translated along the axis of the patient (the z-axis). Alternatively, the patient table may be translated with respect to the C-arm. The C-arm also may be capable of being rotated about the z-axis. The x-ray source of the C-arm imaging systems may be modeled as projecting a cone of x-ray radiation through a volume of the patient and subsequently detected by the detector.

The C-arm imaging systems have been used to generate three dimensional reconstructions of volumes within patients. The C-arm imaging systems usually rely on partial circle scans over an angular interval of 180 degrees plus a cone angle within a single plane. The total angular interval for the partial circle scan typically ranges up to 200 degrees. Alternate imaging systems may be used.

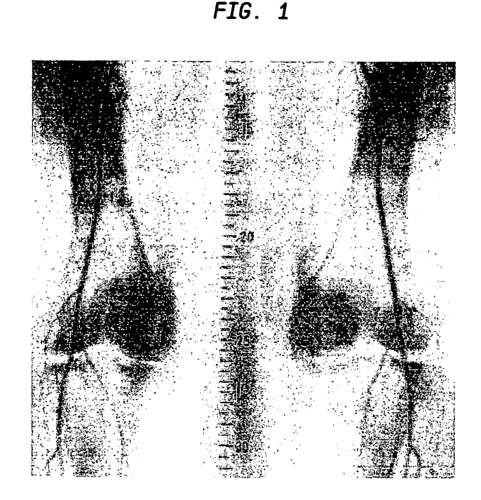

FIG. 1 illustrates a typical first image that may be acquired by the imaging system. The first image in the example shown is of a blood vessel. Additional images may be acquired after repositioning the patient with respect to the imaging system or the imaging system with respect to the patient. Conventional methods may require that the imaging system operates in a fluoroscopic mode while repositioning the patient with respect to the imaging system, or vice versa, to properly reposition the patient and/or the imaging system for the next image. However, fluoroscopic operation exposes the patient to continuous low level radiation during repositioning.

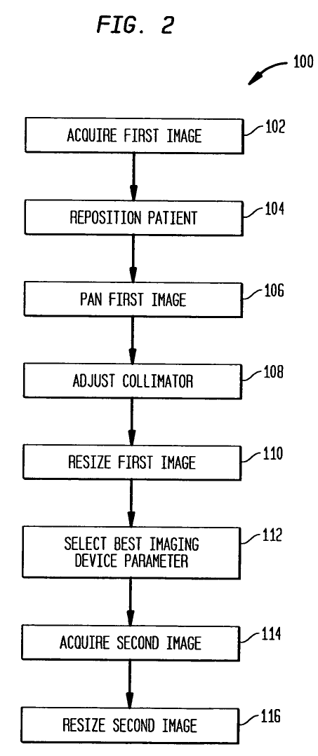

FIG. 2 illustrates a flow chart of a method 100 of reducing the exposure of a patient to radiation during an x-ray imaging procedure. The method 100 may include acquiring a first image 102, repositioning a patient or a component of an imaging system with respect to the other 104, automatically panning the first image 106, adjusting a collimator 108, automatically resizing the first image 110, automatically selecting an imaging device parameter 112, acquiring a second image 114, and automatically resizing the second image 116. The method 100 may include additional, fewer, or alternate acts, such as not performing one or more of acts 106, 108, 110, 112 and/or 116.

A first image of a region of interest internal to a patient may be acquired 102 using an x-ray imaging system. The imaging system may include moving or stationary components and/or one or more sources of radiation. For instance, the source of radiation may be at one end of a movable C-arm device. The patient may be positioned in a first position with respect to the imaging system and/or the source of radiation. The first position may include the patient lying on a table, and the table may or may not be movable with respect to the imaging system. While the patient is positioned in the first position, such as lying on the table, a radiologist or other medical technician may operate the imaging system to acquire the first image 102 of the patient. The imaging system may display the first image acquired on a display screen, monitor, or other display device.

After the first image is acquired 102, the patient may be repositioned 104 to a second position with respect to the imaging system, or a component thereof. For instance, the medical technician may move or reposition a table upon which the patient is lying. Alternatively, the imaging system, or a component thereof, may be repositioned to a second position with respect to the patient. For example, the C-arm or other movable component of the imaging system may be repositioned with respect to the patient lying on the table. In one embodiment, the movable component of the imaging system includes a source of x-ray radiation. Alternate repositioning of the patient and/or the imaging system, or a component thereof, may be used.

As the patient is being repositioned with respect to a component of the imaging system or a component of the imaging system is being repositioned with respect to the patient 104, the imaging system or a data processor associated with the imaging system may track the current positional relationship between the patient and the component of the imaging system.

For instance, the imaging system or the data processor may contain a map in a memory of a real world coordinate system. The patient may be represented as being at the origin of the coordinate system and the imaging system may occupy an initial position within the coordinate system with respect to the patient. As the imaging system, or the component thereof, such as a C-arm, moves within the coordinate system, the position of the imaging system or the component may be updated on the map. Alternatively, as the patient moves within the coordinate system, the position of the patient may be updated on the map.

In one embodiment, with the source at one end of the C-arm, the imaging system may automatically track and/or calculate the location of the source with respect to a fixed patient or patient table based on the positioning of the C-arm. Alternatively, the imaging system may implement one or more sensors and detectors to track the movement of either the patient, such as a patient lying a table, or the imaging system, or a component thereof, within the coordinate system.

The sensors and detectors may be located on the patient, the table, the imaging system, and/or components of the imaging system. Additionally, the sensors and detectors may automatically determine the initial positioning of the patient with respect to the imaging system. For instance, the imaging system may be positioned along the side of a patient lying on a table (bed-side) or along the front of the table (head-side). Alternate manners of tracking the relative position between the patient and the imaging system may be used.

As the component of the imaging system and/or the patient is being repositioned 104, the first image may be automatically panned 106 or moved across the display in accordance with the change in the positional relationship between the patient and the component of the imaging system. The first image may be automatically panned 106 to match or illustrate on the display the position of the next image to be acquired by the imaging system.

In one embodiment, the imaging system is an x-ray imaging system that exposes the patient to radiation while acquiring images. With the x-ray imaging system, the first image may be automatically panned 106 to illustrate the position of the next exposure of radiation to the patient if an additional image is to be acquired using the x-ray imaging system. Hence, by automatically panning the first image either during and/or after repositioning, the display illustrates the part of the anatomy of the patient that is going to receive the next release of x-ray radiation while the x-ray imaging system obtains the next image.

In other words, after the repositioning of the patient or a component of the imaging system, the method and system determine whether an image of the target area, i.e., the region of interest of the patient, will be acquired with the next image taken by the x-ray imaging system. Typically, an x-ray imaging system exposes a patient to radiation while acquiring an image. If the x-ray imaging system will not acquire an image of the region of interest for a given positioning between the patient and the imaging system, the method and system may notify the medical technician. As a result, the patient or the imaging system may be further repositioned by the medical technician before acquiring the next image and exposing the patient to another dose of radiation, such that the next image acquired will more likely include an image of the desired region of interest. The proper position is identified with only the first image or based on an iterative process with fewer images than otherwise used for positioning.

For instance, if a patient is repositioned too far with respect to the imaging system or vice versa, the first image may be automatically panned off of the display. If the first image is automatically panned off of the display, an arrow indication may be generated on the display. The arrow indication indicates the direction by which the region of interest has moved with respect to the display and the imaging system, or a component thereof, such as a component having the source of x-ray radiation. The region of interest may be moved back into view on the display by moving the imaging system, or the component thereof, in the direction of the arrow.

If, after the first image has been automatically panned in response to the repositioning, the first image remains visible on the display, the medical technician may adjust a collimator 108. The collimator may have any number of blades or other components that adjust the coverage area, coverage shape and/or direction of one or more beams of radiation. For instance, opening one or more collimator blades may increase the coverage area of a beam of radiation that a patient is exposed to and closing one or more collimator blades may decrease the coverage area of a beam of radiation that a patient is exposed to.

After the medical technician adjusts the collimator 108 to select a coverage area, the first image may be automatically resized on the display 110. The first image may be automatically zoomed or expanded on the display to fill the screen if the collimator and/or one or more collimator blades have been more closed. Alternatively, the first image may be automatically shrunk if the collimator and/or one or more collimator blades have been more opened. Automatically resizing the first image 110 on the display may include automatically expanding the first image if the selected coverage area of exposure of the collimator decreases and automatically shrinking the first image if the selected coverage area of exposure of the collimator increases.

As the coverage area of the collimator is adjusted, an imaging device parameter may be automatically selected 112 based upon the coverage area selected. The imaging device is an image acquisition device. The imaging device parameter automatically selected is a parameter or setting on the imaging device of the imaging system that enhances the visual quality of the image shown on the display of the imaging device for the selected coverage area. The imaging device parameter automatically selected may result in approximately the ‘best’ or optimal visual quality for the image being displayed on the imaging device. Automatically selecting an imaging device setting may alleviate the need for the medical technician to manually select an imaging device setting based upon the selected coverage area and/or in response to the image viewed on the display.

In one embodiment, the imaging system may include a detector operating as an imaging device. The detector may be an image intensifier detector or a flat panel detector, both of which have settings for various parameters for improving the quality of the image displayed for a given coverage area of the collimator and/or other variables. The image intensifier detector is an x-ray detector in which x-rays produce electrons that are accelerated and focused by an electric field to strike a phosphor screen. The resulting optical photons form an amplified map of the original x-ray distribution. On the other hand, the flat panel detector is a digital detector that may permit high definition digital fluoroscopy and radiography. Alternate detectors and imaging devices may be used.

With an image intensifier detector, the imaging device parameter automatically selected may include a ‘best’ or optimal image intensifier parameter that enhances the quality of the image displayed based upon the selected coverage area of the collimator or other variables. Alternatively, with a flat panel digital detector, the imaging device parameter automatically selected may include a ‘best’ or optimal detector zoom parameter that enhances the quality of the image displayed based upon the selected coverage area or other variables. Alternate imaging device parameters may be automatically selected.

In one embodiment, the imaging device parameter controls the “mode” of the detector. The detector mode informs the detector of the dimensions of the image that is to be sent to the imaging system, the defect characteristics of the detector, and how to construct the image out of the native pixels of the detector. For example, the pixels may be combined to produce a large area, low resolution image, or the pixels may be used to produce a small area, high resolution image.

A second image of the region of interest internal to the patient may be acquired 114 using the imaging system. The imaging system may automatically resize the second image 116 to substantially fill the display. For instance, the imaging system may automatically expand or shrink the second image based upon the selected coverage area of the collimator. The imaging system may use bilinear interpolation or other techniques to automatically resize the second image such that area of the patient exposed to radiation approximately fills the display or a section of the display.

An electronic shutter may be superimposed over the second image after the second image has been automatically resized. The electronic shutter may be a virtual illustration of the current boundaries of the coverage area of the collimator. For example, the coverage area of the collimator may be non-square, have any shape associated with a multi-leaf collimator or be rectangular, such that the boundaries of the coverage area will not approximately coincide with a square display screen. The electronic shutter may include vertical and/or horizontal lines superimposed upon the display to demonstrate the vertical and/or horizontal boundaries of the coverage area. Alternate electronic shutters may be used.

The method and system may reduce radiation exposure to a patient during a radiographic procedure. The type of radiographic procedure may include one of many forms of angiography, such as cerebral angiography, extremity angiography, renal angiography, pulmonary angiography, lymphangiography, right and left heart ventriculography, coronary angiography, aortic angiography, eye angiography, and cardiac catheterization. Additional, fewer, or alternate angiographic procedures may be supported by the method and system.

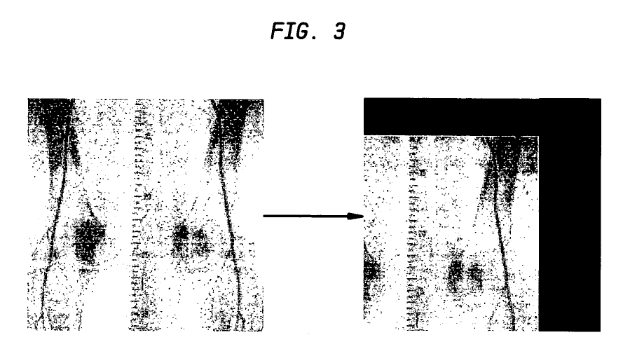

FIG. 3 is an exemplary automatic panning of a first image acquired by the imaging system. As shown on the left hand side of FIG. 3, a first internal image may be obtained from an imaging system and subsequently displayed on a display. In one embodiment, the imaging system is an x-ray imaging system that may include a detector, an x-ray source, a collimator, and a C-arm. The imaging system may include additional, fewer, or alternate components.

The image shown on the left hand side of FIG. 3 is that of a blood vessel. Images of other items or bodily parts also may be obtained. For instance, the region of interest may contain one or more tumors. Also, the region of interest, such as the blood vessel illustrated, may be associated with any area of the body, including the heart, head, neck, abdomen, arms, legs, lungs, kidneys, or other body area.

With the x-ray imaging system, the source of radiation may be secured after acquiring the first image such that the patient is not exposed to radiation until a second image is to be acquired. Hence, the x-ray imaging system is not required to be operated in fluoroscopic mode during the time between acquiring a first and a second image of a region of interest and/or during repositioning.

After the first image is acquired, a medical technician may reposition the patient or a component of the imaging system with respect to the other. For instance, the medical technician may move a table on which the patient is lying or a detector located on a C-arm. The imaging system may track the repositioning of the component of the imaging system with respect to the patient or the patient with respect to the component. For example, the imaging system may track the movement of the table with respect to the imaging system and/or a C-arm or a C-arm with respect to the patient and/or the table. Alternate repositioning may be tracked and monitored.

As a result, as shown on the right hand side of FIG. 3, the imaging system automatically pans the first image across the display to an estimated position of the next exposure based upon the repositioning of the patient and/or a component of the imaging system with respect to the other, such as the movement of a patient table, a C-arm, or other imaging system component. For rotational movement, the first image may be panned by rotating a perspective of the first image, such as associated with viewing a two-dimensional surface with three dimensional rendering. Alternatively, the first image is a three dimensional rendering and the rendering is rotated for panning. After repositioning the patient and/or the component of the imaging system with respect to the other, it may be desirable to have the entire first image remain visible on the display. A first image that remains fully visible on the display is an indication that the entire region of interest will be illustrated in the next image acquired of the region of interest by the imaging system.

If the amount of repositioning results in the first image being automatically panned off of the display, an arrow indication may be drawn on the display. The arrow indication may indicate the direction that the region of interest has been repositioned with respect to the display and/or a component of the imaging system. By further repositioning the component of the imaging system in the direction that the arrow points or further repositioning the patient in the opposite direction that the arrow points, or vice versa, the region of interest may again fall within the coverage area of a collimator and the first image may again become completely visible on the display.

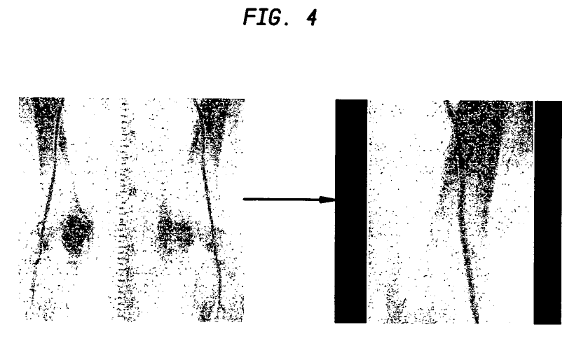

FIG. 4 illustrates automatically resizing the first image based upon an adjustment of a collimator. After repositioning the patient with respect to the imaging system, or vice versa, the medical technician may adjust the coverage area of the collimator, such as by one or more adjusting collimator blades or other collimator components to redirect or alter the size of a beam of x-ray radiation. The displayed first image may be automatically zoomed or expanded to substantially fill the display as the coverage area of the collimator decreases. On the other hand, the displayed first image may be automatically shrunk as the coverage area of the collimator increases.

The example of FIG. 4 illustrates expanding the first image to substantially fill the display as the coverage area of the collimator decreases. In the example shown, the “collimator graphics,” or white lines, are displayed to illustrate that the coverage area of the collimator is rectangular and does not necessarily directly coincide with the approximately square or other shaped display screen. In other words, the white lines are a virtual representation of the physical collimator boundaries. With the example of FIG. 4, the collimator graphics are vertical lines. However, the collimator graphics may be in any direction to match the shape of the collimator. The imaging system may utilize the area of the display outside of the collimator graphics, i.e., the area of the screen other than the region of interest, to display information and instructions to the medical technician.

As noted above, the imaging system may include a detector or other imaging device. The detector may be an image intensifier detector or a flat panel detector, both of which have settings for various parameters for improving the quality of the image displayed for a given coverage area of the collimator or other variable. As the coverage area of the collimator is increased or decreased, the imaging system may automatically select an optimal image intensifier setting associated with the image intensifier detector or an optimal detector zoom stage associated with a digital detector. The imaging system also may automatically change the image intensifier setting or detector zoom stage as necessary during operation. As a result, the need for the medical technician to manually select a ‘best’ or optimal image intensifier setting or zoom stage based upon the selected coverage area or other variable may be eliminated.

After an adjustment of the collimator, the medical technician may acquire the next image. The imaging system may automatically resize the next image acquired using bilinear interpolation such the next image of the region of interest, i.e., the region of anatomy exposed to x-ray radiation, substantially fills the screen.

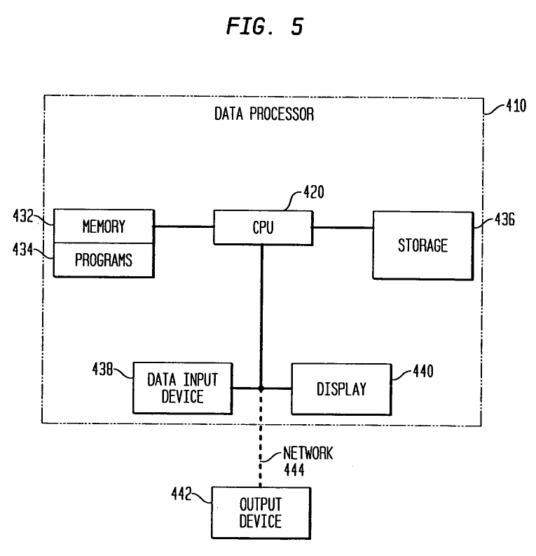

FIG. 5 illustrates an exemplary data processor 410 configured or adapted to be part of the imaging system. The data processor 410 may include a central processing unit (CPU) 420, a memory 432, a storage device 436, a data input device 438, and a display 440. The processor 410 also may have an external output device 442, which may be a display, a monitor, a printer or a communications port. The processor may have additional, fewer, or alternate components.

The processor 410 may be an x-ray system, a detector system, a personal computer, work station, pictorial archiving and communication system (PACS) station, or other medical imaging system. The processor 410 may be interconnected to a network 444, such as an intranet, the Internet, or an intranet connected to the Internet. The data processor 410 is provided for descriptive purposes and is not intended to limit the scope of the present system.

A program 434 may reside on the memory 432 and include one or more sequences of executable code or coded instructions that are executed by the CPU 420. The program 434 may be loaded into the memory 432 from the storage device 436. The CPU 420 may execute one or more sequences of instructions of the program 434 to process data. Data may be input to the data processor 410 with the data input device 438 and/or received from the network 444. The program 434 may interface the data input device 438 and/or the network 444 for the input of data. Data processed by the data processor 410 may be provided as an output to the display 440, the external output device 442, the network 444, and/or stored in a database. The program 434 and other data may be stored on or read from machine-readable medium, including RAM, cache or secondary storage devices, such as hard disks, floppy disks, CD-ROMS, and DVDs; electromagnetic signals; or alternate forms of machine readable medium, either currently known or later developed.

The data processor 410 may control the imaging system that reduces radiation exposure to a patient during an x-ray imaging procedure. The data processor 410 may run a software application or program 434 that performs a number of operations related to the x-ray imaging procedure. Alternatively, the data processor 410 provides user output without controlling the x-ray imaging.

The data processor 410 may direct an imaging system to acquire a first image of a region of interest. The first image acquired may be received by the data input device 438 or the network 444 and stored in the memory 432 or the storage 436. The data processor 410 may direct that the first image be displayed on the display 440, the output device 442, other output device and/or stored.

During repositioning of the patient and/or the imaging system with respect to each other, the data input device 438 or another input device may monitor the relative position between the patient and/or the imaging systems, or a component thereof. The CPU 420 may track the relative position of the patient with respect to the imaging system, or a component thereof, during repositioning.

By tracking movement of the patient and/or imaging system with respect to each other, the CPU 420 may determine the region of the patient that will be exposed to radiation while acquiring the next image, given the current positional relationship between the patient and the imaging system, or a component thereof. Additionally, during repositioning, the data processor 410 may automatically pan the first image across the display 440 or other output screen according to the movement of the patient with respect to the imaging system or vice versa.

Subsequently, the coverage area of a collimator may be adjusted. In response to the adjustment of the coverage area, the data processor 410 may automatically resize the first image on the display 440 or other screen. If the coverage area increases, the data processor 410 may decrease the size of the first image shown on the display 440, the output device 442, or other output device. If the coverage area decreases, the data processor 410 may increase the size of the first image shown on the display 440, the output device 442, or other output device.

The data processor 410 also may automatically adjust the settings for a detector or other imaging device based upon the coverage area such that the quality of the images displayed on the display 440, the output device 442, or other output device is improved for the current variables. For example, the data processor 410 may automatically select an image intensifier parameter if the imaging device is an image intensifier. Alternatively, the data processor 410 may automatically select a detector zoom stage if the imaging device is a digital detector. Alternate detectors and imaging devices may be used.

The data processor 410 may direct the imaging system to acquire a second image. The second image may be obtained via the data input device 438, the network 444, or other input device. The second image may be displayed on the display 440, output device 442, or other output device. Subsequently, the data processor 410 may automatically resize the second image such that it substantially fills the display 440, output device 442, or other output device.

While the preferred embodiments of the invention have been described, it should be understood that the invention is not so limited and modifications may be made without departing from the invention. The scope of the invention is defined by the appended claims, and all devices that come within the meaning of the claims, either literally or by equivalence, are intended to be embraced therein.

It is therefore intended that the foregoing detailed description be regarded as illustrative rather than limiting, and that it be understood that it is the following claims, including all equivalents, that are intended to define the spirit and scope of this invention.

What is claimed:

1. A method of reducing the exposure of a patient to radiation during an x-ray imaging procedure, the method comprising:
acquiring a first image of a region of the patient using an imaging system;
repositioning the patient with respect to a component of the imaging system or the component of the imaging system with respect to the patient;
automatically panning the first image on a display based upon the repositioning of the patient or the component of the imaging system; and
acquiring a second image of the region using the imaging system.

2. The method of claim 1, comprising:
adjusting a collimator to select a selected coverage area of the collimator; and
automatically resizing the first image on the display based upon the selected coverage area.

3. The method of claim 2, wherein automatically resizing the first image on the display includes expanding the first image if the selected coverage area decreases or shrinking the first image if the selected coverage area increases.

4. The method of claim 1, comprising;
adjusting a collimator to select a selected coverage area of the collimator; and
automatically selecting an imaging device parameter based upon the selected coverage area that enhances the quality of the first or second image as shown on the display.

5. The method of claim 4, wherein automatically selecting an imaging device parameter includes automatically selecting an image intensifier parameter.

6. The method of claim 4, wherein automatically selecting an imaging device parameter includes automatically selecting an optimal detector parameter.

7. The method of claim 1, comprising automatically resizing the second image such that the region substantially fills the display.

8. The method of claim 7, comprising using bilinear interpolation to resize the second image.

9. The method of claim 1, comprising drawing an arrow indication on the display if the first image has panned off of the display, the arrow indication indicates the direction by which the region has moved with respect to the display.

10. The method of claim 1, comprising:
selecting a selected coverage area of radiation of the imaging system; and
drawing a virtual representation of the selected coverage area on the display.

11. The method of claim 1, comprising securing an x-ray source during the repositioning of the patient or the component of the imaging system.

12. A method of reducing the exposure of a patient to radiation during an x-ray imaging procedure, the method comprising:
acquiring a first image of a region of the patient using an imaging system;
repositioning the patient with respect to a component of the imaging system or a component of the imaging system with respect to the patient;
selecting a selected coverage area of radiation of the imaging system;
automatically resizing the first image on a display based upon the selected coverage area; and
acquiring a second image of the region using the imaging system.

13. The method of claim 12, wherein automatically resizing the first image on the display includes expanding the first image if the selected coverage area decreases or shrinking the first image if the selected coverage area increases.

14. The method of claim 12, comprising automatically panning the first image on the display based upon the repositioning of the patient or the component of the imaging system.

15. The method of claim 14, comprising drawing an arrow indication on the display if the first image has panned off of the display, the arrow indication indicates the direction by which the region has moved with respect to the display.

16. The method of claim 12, comprising drawing a virtual representation of the selected coverage area on the display.

17. The method of claim 12, comprising automatically selecting an optimal imaging device parameter based upon the selected coverage area.

18. The method of claim 17, wherein automatically selecting an optimal imaging device parameter includes automatically selecting an optimal image intensifier parameter or an optimal detector parameter.

19. The method of claim 12, comprising automatically resizing the second image using bilinear interpolation such that the region substantially fills the display.

20. The method of claim 12, comprising securing an x-ray source during the repositioning of the patient or the component of the imaging system.

21. A data processing system for reducing the exposure of a patient to radiation during an x-ray imaging procedure, the system comprising:
a display operable to show a first image of a region of the patient obtained using an x-ray imaging procedure; and
a data processor connected with the display operable to direct panning or resizing of the first image on the display based upon the movement of the table or the C-arm.

22. The data processing system of claim 21, wherein the data processor resizes the first image based upon an adjustment of a coverage area of a collimator.

23. The data processing system of claim 22, wherein the display shows a second image of the region obtained using the x-ray imaging procedure after the adjustment of the coverage area and the data processor resizes the second image to substantially fill the display.

24. The data processing system of claim 22, wherein the data processor expands the first image if the coverage area decreases or shrinks the first image if the coverage area increases.

25. The data processing system of claim 22, wherein the data processor selects an optimal imaging device parameter based upon the adjustment of the coverage area.

26. A computer-readable medium having instructions executable on a computer stored thereon, the instructions comprising:
displaying a first image of a region of a patient obtained using an imaging system on a display;
panning the first image on the display in response to movement of a table or a C-arm;
resizing the first image on the display in response to an adjustment of a coverage area of a collimator; and
displaying on the display a second image of the region obtained using the imaging system after the adjustment of the coverage area.

27. The computer-readable medium of claim 26, wherein resizing the first image on the display includes expanding the first image if the selected coverage area decreases or shrinking the first image if the selecting coverage area increases.

28. The computer-readable medium of claim 26, the instructions comprising resizing the second image such that the region substantially fills the display.

29. The computer-readable medium of claim 26, the instructions comprising drawing an arrow indication on the display if the first image has panned off of the display, the arrow indication indicates the direction by which the region has moved with respect to the display.

30. The computer-readable medium of claim 26, the instructions comprising selecting an optimal imaging device parameter for the coverage area.
